(19) United States
(12) Patent Application Publication
(10) Pub. No.: US 2016/0206476 A1
(43) Pub. Date: Jul. 21, 2016

(54) EYE COVER DEVICE

(71) Applicant: OCCLES LTD, Edinburgh (GB)

(72) Inventors: Hugh Robertson, Edinburgh (GB); Lynne Mcleod, Edinburgh (GB)

(21) Appl. No.: 14/915,555

(22) PCT Filed: Aug. 29, 2014
(86) PCT No.: PCT/GB2014/052632

§ 371 Date: Feb. 29, 2016

(30) Foreign Application Priority Data
Aug. 29, 2013 (GB) 1315424.0

(51) Int. Cl. A61F 9/04

(52) U.S. Cl. CPC A61F 9/045

(57) ABSTRACT

Many people like to spend time in the sun, in particularly sunbathing. The risks of exposure to the sun are well documented, such as aging of the skin caused by ultraviolet light rays and development of skin pigmentation. People are encouraged to take steps to protect their skins and eyes from exposure to the sun, particularly delicate areas of skin such as the periorbital area around the eyes. Typically a person wears sun glasses to protect their eyes and a number of designs are available that successfully protect a user's eyes from harmful rays from the sun or artificial rays from a sunbed however; they can lead to unsightly tan lines particularly at the temples and across the bridge of the nose. The present invention provides a bridgeless eye cover device for covering the delicate skin around the eyes, preventing or limiting transmission of light and reducing unsightly tanning lines.

### FIELD OF THE INVENTION

The present invention relates to an eye cover device, particularly but not exclusively for protection from the sun.

### BACKGROUND

Many people like to spend time in the sun, in particular sunbathing. The risks of exposure to the sun are well documented and people are encouraged to take steps to protect their skin and eyes from exposure to the sun. In particular skin can be damaged by ultraviolet light rays leading to premature aging of the skin and pigmentation.

Typically a person wears sunglasses to protect their eyes. A number of designs are available that successfully protect a user's eyes from harmful rays from the sun or artificial rays from a sunbed however they can lead to unsightly tan lies particularly at the temples and across the bridge of the nose. Furthermore the sunglasses may not fully protect the delicate periorbital skin.

Some sunglasses have been adapted to have narrow arms and bridge sections to reduce this problem however this does not fully eliminate the problem.

Modified screens, glasses and goggles have been developed for artificial sunbeds however these are often not suitable for outdoor or prolonged use. For example, stick on eye covers do not last very long and cannot be easily refitted therefore a new pair of such covers would be required each time an adjustment is made, which would be costly and the continual use of adhesive products on delicate eye area is undesirable.

Sunbed goggles have also been developed to only cover the eye socket and are typically connected by elastic. However, the elastic can be very uncomfortable if worn for long periods of time and is also unsightly. Prior Art

Accordingly a number of patent applications have been filed in an attempt to resolve the problem or similar, including the following:

U.S. Pat. No. 7,931,366 (LANDO) discloses eyewear, comprising: a pair of lenses; a pair of releasable connectors connected to respective inner ends of said lenses, said connectors being securely connectable together and releasable from each other; a pair of temples connected to respective outer ends of said lenses; and a strap connected between rear ends of said temples.

United States patent application US 2004 207 805 (CHOVAN et al) discloses protective eyewear comprising: first and second eyepieces formed of uniform material, wherein said first and second eyepieces each comprise an eye cup and a lens integrally formed with said eye cup to comprise a unitary eye cup/lens combination; and a nose bridge removably coupled to each of said first and second eyepieces via an attachment means, said nose bridge comprising first and second supports and an elevated rise that reduces blockage of ultraviolet light from the nose and brow area of a wearer when attached.

International patent application WO-A-03 085 444 (MEMBRINI) discloses glasses with a frame made of an elastic clamp wearable on the skull of the user and whose back jaw is made for pressing in moderation on the nape, while the front jaw is made for pressing in moderation on the front, keeping the lenses overhung in front of the eyes.

U.S. Pat. No. 5,956,117 (SUH) discloses an eyeglass assembly, comprising: a left lens element for covering at least a portion of the left eye; a right lens element for covering at least a portion of the right eye; a temple element having a first end coupled to said left lens element and a second end coupled to said right lens element.

U.S. Pat. No. 4,129,362 (LORENZO) discloses eyeglass structure including a first and a second lens holding ring and a lens in each ring, said lens holding rings each including a portion extending toward the other lens holding ring and comprising mutually inter-cooperating means for interconnection in male and female relationship above the nose level of a wearer, and a pair of temple pieces, each temple piece being pivotally connected to the lens holding rings and each terminating at a distal end zone and a band segment of spring material having a first end connected to the distal end zone of one of the temple pieces and a second end connected to the distal end zone of the other of the temple pieces at a pivotal connection, said band segment being sized to fit snugly about the head of a wearer to distribute the entire weight of the eyeglass structure to the head and not to the ears and nose, and cushions connected at the juncture of the band segment and temple pieces and confronting one another for distributing tension loads by the band segment.

U.S. Pat. No. 4,154,513 (GOULDEN) discloses a sunbather's eye protective device comprising a pair of generally disc shaped opaque eye covers sized to cover the eyes and eyelids of a wearer, a frame comprising a crossbar and temple pieces pivotably attached to opposite ends of said crossbar, and means for adjustably mounting said eye covers on said crossbar so that they are movable in the vertical and horizontal directions, said frame, mounting means and all parts other than the eye covers being made of synthetic resinous material which is substantially transparent to the tanning rays of the sun.

Japanese Patent Application JP2003078982 A discloses a headphone with glasses by which a user can listen to music or the like while using the sunglasses.

U.S. Pat. No. 5,009,496 reveals an eyeglasses and headgear combination is disclosed. The eyeglasses may be worn alone or in combination with the headgear. The temple member of the eyeglasses is alternatively positionable behind the ear of the user or attached to the headgear.

In contrast the present invention provides a means of ensuring that a user is tanned across their face with minimal tan lines and minimal risk to eyes.

### SUMMARY OF THE INVENTION

According to the present invention there is provided an eye cover device comprising: a pair of screens, each screen adapted to cover an eye, both screens connected to an arm that passes around a rear portion of a user's head. The screens may be displaceable by means of an arm mechanism.

In this preferred embodiment the screens are connected one to another by means of the arm and have no portion that extends over the wearer's nose, unlike with conventional eyewear. As a result of the arm the eye cover device, or device, not passing over the wearer's nose, the eye covers are therefore bridgeless. This reduces occurrences of tan lines forming over the nose due to eyewear and can allow improved circulation of air around the eyes thus reducing perspiration in hot conditions.

In preferred embodiments both screens are joined by means of a single arm. Typically the arm is a curved elongate length of material arranged in use to pass around the back of the user's head with a screen attached to distal ends of the arm.

Preferably the arm is curved in such a way as to match back and sides of the user's head.

Preferably the arm is arranged to pass over the user's ears so as to be positioned on the user's head at a fixed position in a similar manner as to standard eyewear. The arm may include arches for passing over and resting upon the user's ears.

Ideally the arm is dimensioned to follow the curvature about the ear and to drop downwards behind the ear to sit at, or close to the nape of a neck. Preferably the arm sits in around the wearer's hairline so as not to create a tan line if the user is sunbathing face down. In this way the arm may also be hidden by the hair in use, therefore being more discreet.

The arm may comprise two side portions and a rearward portion, the rearward portion of the arm being inclined downwardly relative to the side portions. The side portions may be substantially straight when viewed from the side. The side portions may be curved when viewed from above.

Ideally a rearward portion of the arm includes a dropped portion that, when worn, extends downwards from the ear to the nape of the neck. In this way the arm does not dig in to the user's head if the user is resting their head on a surface such as a headrest. Additionally if the user has long hair the hair can be readily arranged over the arm rather than having to part the hair or push it away comfortably to accommodate the arm.

Preferably the arm is sprung so as to be biased to a curvature that matches the shape of a typical users head. Typically the arm is an open loop wherein distal ends finish at or near to the user's temple.

Ideally the arm is dimensioned to contact both sides of the user's head. Preferably the arms contact the head about the ear and/or behind the ear so as to exert a gentle pressure by means of the sprung arm that serves to hold the arm and thereby the screens in position.

Therefore the arm touches the side of the user's head and the screens contact the user around the eyes so there is no contact over the temples. Typically the arm may be flexed in order to remove the screens from the user's eyes, therefore providing one arm mechanism for displacing the screens.

In some embodiments the arm length may be adjustable. The arm may have telescopic or displaceable portions so as to allow the arm length to be altered. This allows a user to move the screens closer to or further from the eyes, thereby ensuring the screens are correctly positioned over the eyes of different users.

Advantageously adjustment of arm length enables the screens to be removed from the eyes and therefore the eye covers to be removed from the user. This provides a second arm mechanism for displacement of the screens.

The arm may be produced in different lengths for different classes of users, for example in different lengths for adult and child users. Similarly the screens may be produced in different sizes for different classes of users, for example adults or children. In some embodiments, the range of adjustment of the arm may be such that the same design may be used for both adults and children.

In some preferred embodiments each screen has one or more elongate hollow member(s) arranged on an outer edge of the screen which extends perpendicular to the screen towards the user's ear. Therefore the screens are arranged in the correct orientation to cover the eyes.

In use the hollow member(s) receive(s) the distal ends of each arm. In this way the arm may be inserted into the hollow member by the desired length which ensures the screens cover the eyes. Furthermore, displacement of the screens from the eyes in this way allows the device to be more readily removed from the user's head or lowered to around the user's neck by extending the arms.

Preferably the arm may be ridged at the distal end so as to be capable of incremental adjustment in use by being adjusted one ridge at a time into the hollow member.

Ideally the ridged region is arranged in front of the ear, traversing the temple so as to not to cause discomfort to the ear when worn.

Ideally it may be possible to detach the hollow member and thereby screen from the arm so that it may be replaced or more readily stored when not in use.

In some embodiments the arm may include a bulbous distal end to prevent the arm being fully detached from the hollow member.

Typically the hollow member may include a catch to hook each ridge and therefore enabling incremental adjustment.

Ideally the hollow member may include a cutaway section so that the user can see position of the arm within the hollow member, in this way the user may be able to ensure both arms are independently adjustable.

In some embodiments the hollow member may include increments, for example numbers, to guide a user to the length of the arm.

In an alternative embodiment the distal end of the arm may be hollow so as to receive a concentric member arranged on or integrated with the screen. The concentric member may be ridged and include a cutaway section in a similar manner to the previously described arm so as to be incrementally adjusted.

Typically adjustable portions on each side of the arm, are located near to or at the distal end of an arm so that the length can be easily adjusted in use and so as not to be located at the back of the head which may cause discomfort when the user is resting their head on a headrest, for example if lying down on a sunbed.

In further embodiments the, or each, arm may be divided or dividable into a plurality of parts, for example the arm(s) may be formed from two halves or two arm sections and a third rear part, wherein the parts can be connected in use to form the device but may be readily separated. In this way parts may be exchanged and/or replaced. Advantageously this allows the device to be readily packed away for storage or transport.

Typically the arm is formed from a length of lightweight, flexible, resilient, water resistant material such as plastic, carbon, rubber, metal, metal alloy or an elastomer. For example the arm may be formed from an injection moulded plastic or may be 3D printed.

In some embodiments the arm or part of the arm is formed in a dual layer format such as a bimetallic material wherein one layer is able to contract or retract at a faster rate than another in response to a temperature change. For example the arm, or part of the arm, may be caused to wrap more tightly around the user's head in response to heat produced by the user and heat from the sun. Therefore the device becomes more securing attached to the user due to increased temperature.

In some embodiments the arm and or screens may be formed from a synthetic material with colour changing capabilities. For example a material that changes colour in response to temperature or after a predetermined period of time. In this way the user or a third party can identify when a set period of time has lapsed or if temperature has exceeded an optimal level or range. When this is detected the user may choose for example to move away from direct sunlight, for example moving into the shade, and/or apply more sun cream. Therefore the colour change serves as a warning.

In preferred embodiments the arm is formed from a resiliently deformable material so as to be capable of flexure when being positioned on the user and whilst worn without causing damage to the arm. For example the screens may be pulled apart by flexing the arms in order to be positioned about the user's head wherein the screens are parted and once in position the arm returns to its original shape allow the screens to be placed over the eyes, therefore further aiding with addition and removal of the device.

In the preferred embodiment each screen is sized and shaped to only cover the user's eye as defined by the eye socket to include the eyelids and caruncle, enabling maximum skin exposure to the sun and protection to the most delicate areas of the eye. In particular each screen is dimensioned to cover the delicate periorbital skin, including the eyelids so as to limit/prevent sunlight damage to the eyes.

The screens may be produced in different sizes, for example adult and child sizes.

Ideally each screen is substantially oval shaped so as to match the eye and to ensure coverage of the delicate parts of the eye including periorbital skin.

In some other embodiments the screens may be teardrop shaped so as to extend and taper towards the arm, therefore providing further protection to delicate skin surrounding the eye and tapering to a tip to which the arm may be attached.

Typically, for each screen, the tapered portion of the screen may attached to or be integrated with the hollow member.

Preferably each screen is arcuate, typically arching over the eye so as not to interfere with the user's eyelashes or eyelids when worn and therefore allowing the user to blink without their eyelids being obstructed.

Ideally the screens do not allow transmission of harmful light that may be damaging to the eyes and surrounding delicate skin.

In other embodiments the screens may allow light to penetrate through and for example may provide protection from some light such as ultraviolet (UV) light. In such embodiments the screens may comprise lenses that may be transparent or translucent so as to permit the user to see through them.

In another embodiment the screens may comprise a lens such as a prescription lens. For example the lens may be formed from optical glass.

In other embodiments the device may include a plurality of lenses in cooperation or combination, for example wherein the lenses are arranged in sets and can be selected by the user, such as a clear lens, photochromic or tinted lens. Lenses may be displaceable and removable so as to enable them to be changed by the user.

Typically the screens may be formed from an injection moulded plastic.

In some embodiments exterior faces of the screens may be made available in a number of designs. For example the exterior face of each screen may be coloured and/or patterned.

In other embodiments stickers may be provided for addition to the screens in order to personalise the screens. In some embodiments stickers may be added to transparent or translucent screens to alter the amount of light that can pass through or to prevent light from passing through. Alternatively or additionally the stickers may be coloured so as to tint the screens, for example for use with light therapy treatments wherein light or a particular colour(s) may have health benefits.

In preferred embodiments the screens may be resiliently deformable so as to be capable of specifically fitting to the user's eyes by being able to be readily flexed when fitted and worn, therefore providing improved comfort and wearability.

Typically the screens will be scratch resistant thus not requiring use of a cover or case at all times.

The screen may include or be associated with the peripheral portion. In preferred embodiments each screen has a peripheral portion, cowl or pad of soft resilient material on an inner side of the lens that will ensure a comfortable connection with the eye of the user and minimising peripheral introduction of light. Therefore the screen contacts with the eye to form a barrier preventing light entering the eye of passing to the surrounding delicate skin.

Preferably the peripheral portion, cowl or pad, here on in referred to as the peripheral portion, is fixed against the eye by a suction fit, for example a gentle suction fit, in a similar manner to swimming goggles. In this way the screens are held in position over the eye by means of a peripheral portion.

The peripheral portion preferably also aids in preventing the screen from touching the eye, for example the eyelids or eyelashes. Therefore the peripheral portion serves to distance the screen from the eye.

Typically the peripheral portion is formed from a resiliently deformable material such as rubber, silicone or neoprene so as to be capable of forming a comfortable suction fit when pressed against the skin.

Preferably the peripheral portion is opaque so as to prevent light passing through to the eyes.

In some embodiments the peripheral portion may be transparent or translucent so as to allow some light to pass through to the eyes. For example the peripheral portion may only allow transmission of certain light rays or may be tinted so as to be coloured, for example to aid with light therapy treatments.

For example, for each screen, the peripheral portion comprises a band or loop of silicone arranged around an outer part of the screen so as to form the peripheral portion. Typically the peripheral portion is pressed against the eye in order to create a suction fit which serves to hold the screens against the eyes.

In preferred embodiments the peripheral portion does not form a vacuum when fitted to the eye, but instead includes a channel or breathable piece that allows circulation or air to prevent build-up of moisture, for example due to condensation and/or perspiration.

For example each peripheral portion may include at least one channel traversing the peripheral portion to prevent a vacuum forming and thereby allowing air to circulate.

In another embodiment at least part of the peripheral portion may be formed from a breathable material for example a fabric which allows circulation or passage of air and/or moisture in one or both directions and/or prevents moisture build-up. For example, suitable known breathable fabric may be used.

In some embodiments the peripheral portion may be concertinaed so as to allow the peripheral portion to be collapsed as pressed against the eye and then to be released away from the eyes when no longer pressed by the user.

The peripheral portion may comprise at least one fold, for example a concertina fold. Any suitable number of folds may be used. A channel and/or breathable piece may be located on or near a fold, for example at the base of a concertina fold.

The peripheral portion may comprise an engagement surface for making contact with an eye region of the user. The engagement surface may be generally in the shape of an oval or teardrop-shaped ring. The engagement surface may comprise an inner edge. The inner edge may be substantially oval or teardrop-shaped. The inner edge may comprise a substantially straight portion. The substantially straight portion may be at an end of the engagement surface that, in use, is proximal to the nose of the user.

The engagement surface may comprise an outer edge. The outer edge may be substantially oval or teardrop-shaped. The outer edge may comprise a substantially straight portion. The substantially straight portion may be at an end of the engagement surface that, in use, is proximal to the nose of the user.

For at least one of the screens, optionally each of the screens, the device may comprise means for reducing a difference in pressure between one side of the screen and another side of the screen when in use. When in use, the engagement surface may be engaged with the eye region of the user, forming a suction fit. When engaged, the pressure inside the peripheral portion (e.g. the pressure on the side of the screen that faces the user) may be lower than the pressure outside the peripheral portion (e.g. the pressure on the other side of the screen, which faces away from the user). A pressure difference between the inside of the peripheral portion and the outside of the peripheral portion may be reduced by the provision of a channel and/or breathable piece.

The peripheral portion may comprise a channel and/or a breathable piece. The means for reducing the difference in pressure between one side of the screen and another side of the screen may comprise the or a channel and/or breathable piece.

The depth of the peripheral portion may be varied so as create different distances of the screen from the eye suitable for different users.

Ideally the peripheral portion is bonded to the screen by flexible hard wearing adhesive material so as to prevent dislocation in use.

In further embodiments the peripheral portion may extend beyond the screen footprint so as to further cover the eye without requirement for a larger screen.

In a preferred embodiment the screen and peripheral portion may be integrated as one part, for example being formed from the same material. In this way the screen and peripheral portion could be moulded as a single part in order to save costs.

In some embodiments the peripheral portion may include an eye treatment solution such as a cooling gel, anti-aging cream, moisturiser, anti-inflammatory cream or eye gel. The peripheral portion may include a layer of eye treatment solution or the peripheral portion may be impregnated with the solution. In this way as the device is worn the eyes are treated with the solution so as to improve condition of the eye and surrounding tissue.

The peripheral portion may be detachable and disposable so that it can be removed and replaced after use. Advantageously therefore different peripheries (peripheral portions) may be used depending upon user choice or need. It is important to include that the wearer can sit up, lie face down, reclined or on their side and they will remain in place, securely, with no pinching or discomfort due to design.

In some embodiments the peripheral portion may be heated or heatable so as to be warmed before placing over the eye. For example the peripheral portion may include self-heating means or a controllable heating element so as to alter temperature of the peripheral portion.

Alternatively the peripheral portion may be cooled, for example being placed in a fridge or may include cooling means to sooth the eyes and surrounding area for example aiding to reduce puffiness around the eyes.

The part of the arm that traverses the temples may be shaped so as not to be in contact with the user's temples, and ideally spaced away from the temples sufficiently to allow the temples to be exposed to the sun, so as to prevent obvious tanning lines. Furthermore this portion of the arm may be thinner so as reduce/prevent tanning lines.

In some embodiments the arm is transparent to enable the device to be discreet and allow the user to gain a tan where the connecting arm crosses the temples or other areas of the head not covered by hair or headwear, reducing chances of obvious tanning lines.

In some embodiments the arms may include grips to assist in keeping the device in place. Ideally the grips are arranged on an inner surface of the arm so as to enable the user to wear them, such as behind the point of ear.

In preferred embodiments the arm may be padded along its all or part of its length for comfort. Typically the padded section may comprise a resilient, soft material such as cellular structures, for example neoprene or foam.

The grips may also serve as padding or vice versa.

Further embodiments may include: inflatable bladders arranged on the arm for comfort and to aid with fitting. In some embodiments the bladders may be adjustable so that pressure can be adjusted thereby altering size of the bladder and fit to the user's head. This may also be of use when worn in water so that the eye covers floats and so is easy to retrieve.

In another embodiment layers of padding may be displaceably attached to the arm, for example by hook and eye connection such as Velcro (RTM) thus allowing the user to select the preferred level and type of padding. Advantageously old, worn or dirty padding can therefore be removed, cleaned and/or exchanged.

In preferred embodiments where the arm passes behind the back of the wearer's head, the arm is formed relatively wider. This creates a more comfortable platform for the head to rest when in use and a suitable area for padding.

In some alternative embodiments the screen may be independently separable, so that screen type and/or size can be readily altered and to enable replacement of worn or damaged screens. For example the screen may be attached to the device by a clip, friction fitting or detent mechanism so as to allow ready detachment.

In some alternative embodiments the screens may be hingeably connected to the arm, or to the hollow member so as to allow the screens to pivot and therefore be readily positioned over, and displaced from, the eyes by means of the hinges. Therefore the position of the screen over the eye is user settable for an improved fit against the eye.

Ideally the hinges may include a ratchet so as to be adjusted incrementally to and from the eyes of the user. For example the screens may be displaced from the eyes when the user sits up on a sunbed, if talking to another person or when eating or drinking.

The positioning of the screens by the arm mechanism may be user settable. The position of each screen may be user settable by means of a ratchet.

In some embodiments the eye covers may produce an audible sound such as a click when adjusted.

In some embodiments a cushion may be provided on the arm for comfort. Ideally the cushion is soft being air or liquid filled cushion that when compressed, for example when the user lays back, this causes cushion contents to be forced down the arms to cause the screens to pivot over the eyes. Advantageously the cushion may also serve as padding and/or grips.

Some embodiments of the arm may include small pistons to create movement. For example tubes may run down the arm of the device to carry air or liquid to the pivot points to enable hydraulic movement of the screens.

In some other embodiments a band may be attached to the screens so as to lie inside the arm in use. Typically the band is shorter than the arm so that when the user lays back against the arm, the band is forced against the arm at the rear and the screens are caused to fold inward and thereby against the eyes.

In some further embodiments a positioning strap serves to prevent the arm dropping or falling. Ideally the strap passes over the user's head. Typically the strap is adjustable by buckle, extendible ratchet, hook and loop connection such as Velcro® so as to be fitted to any user.

In some embodiments the strap is adapted to appear as a head band. Alternatively the strap may be transparent so as to be discreet.

Said strap is ideally comprised of a light weight flexible material such as fabric, plastic or silicone.

In other embodiments the device may include an alert that serves to notify the user. The alert may be triggered after a predetermined period of time or on identification of a particular factor such as presence of a person, humidity levels or temperature.

The alert may comprise vibrating means, an audible noise such as beep or visual, such as a flashing light.

Preferably the alert is programmed to be activated when specific conditions are met or after a predetermined time period has passed. For example the alert may be activated after the user has been in the sun for one hour, so as to remind them to apply more sunscreen.

The alert may be programmed by means of a remote unit such as a smartphone, tablet or computer, wherein alert parameters may be set. When the set parameters are met a signal may be sent from the remote unit to the device in order to activate the alert.

Typically the device may include a receiver in order to receive the signal.

Advantageously the vibrating means may provide physical benefits to the eye by massaging the eye and surrounding areas improving circulation to the area such as to enhance lymphatic drainage, puffiness around the eyes and reducing wrinkles.

The vibrating means comprises a vibrator module that is mounted on, or integrated with the device. Typically the vibrator module may be located on the or about the screens so as to causes greatest vibration in this area.

In some other embodiments the vibrator module may be located on the arm.

In yet further embodiments the device may include a sensor for detecting strength of the sun, for example measuring ultraviolet (UV) light to advise the user of when UV light levels may be more harmful. This sensor is linked a transmitter capable of transmitting information from the device to a remote unit, such as a computer, tablet, or smartphone that can display the data obtained by the sensor. For example the data may be collected and interpreted by an application on a smartphone.

Ideally the data is wirelessly transmitted for example by radio frequency, or Bluetooth®. Therefore the device may include a transmitter and a receiver for sending and receiving data. Preferably the transmitter and receiver may be integrated with the device, for example being housed within the arm.

In other embodiments the transmitter and receiver may be removable. In this way the transmitter and receiver may be retro fitted to a device.

In some embodiments the device may include both a sensor and an alert.

In some embodiments the device may include an integrated display means, such as a display screen, or colour changing portion to indicate the data collected by the sensor. In this way there is no requirement for a remote unit. For example the display means may advise the user of UV light levels so as to guide them as to when the sun is strongest and it may therefore not be advisable to be exposed to the sun.

Additionally in some embodiments the device may be capable of providing location information by transmitting a signal to the remote unit, wherein the remote unit has a global positioning system (GPS). Therefore location of where data is collected by the sensor can be logged and optionally recorded. For example the user may measure and subsequently compare UV light levels in different locations or the same location at different times.

In some embodiments the sensor may be linked to the alert so that if for example UV light levels become dangerously high the alert is triggered so as to advise the user.

In other embodiments the sensor may be capable of detecting temperature or a temperature range so that the users may be alerted if temperature is too high or too low.

In another embodiment the device may be capable of downloading and storing audio information such as music or audio books. The device includes earphones so that the user may listen to the audio information whilst wearing the device therefore removing the requirement for additional pieces of equipment to be carried which may be problematic whilst sunbathing for example on a beach and not having safe storage for valuable equipment.

In yet further embodiments the device may include a microphone for recording audio communications such as the user's voice. Typically the device may include a memory means for saving such recordings, such as flash drive, so that the recordings may be replayed at a later time and optionally exported or transferred to another unit capable of playing the recording.

Preferred embodiments of the invention will now be described, by way of example only, and with reference to the Figures in which:

### BRIEF DESCRIPTION OF FIGURES

FIG. 1 shows a top of the one embodiment of the eye cover device; FIG. 2a shows a front view of one embodiment of the eye cover device;

FIGS. 2b to 2e show various views of the eye cover device of FIG. 2a, being worn by a user;

FIG. 3 shows a rear view of one embodiment of the eye cover device;

FIG. 4 shows a side view of one embodiment of the eye cover device;

FIG. 5 shows an isometric view of one embodiment of the eye cover device;

FIG. 6 shows an embodiment of the arm mechanism;

FIG. 7 shows a schematic cross-sectional view of one embodiment of a peripheral portion and screen;

FIG. 8 shows a view of an engagement surface of a peripheral portion of an embodiment;

FIG. 9 shows a further view of an embodiment of the eye cover device;

FIG. 10 shows a view of part of an arm of an eye cover device according to an embodiment.

### DETAILED DESCRIPTION OF FIGURES

The pictured and preferred embodiment of the invention protects eyes from damage caused by light such as ultraviolet (UV) light rays, without creating obvious tanning lines often associated with traditional eyewear.

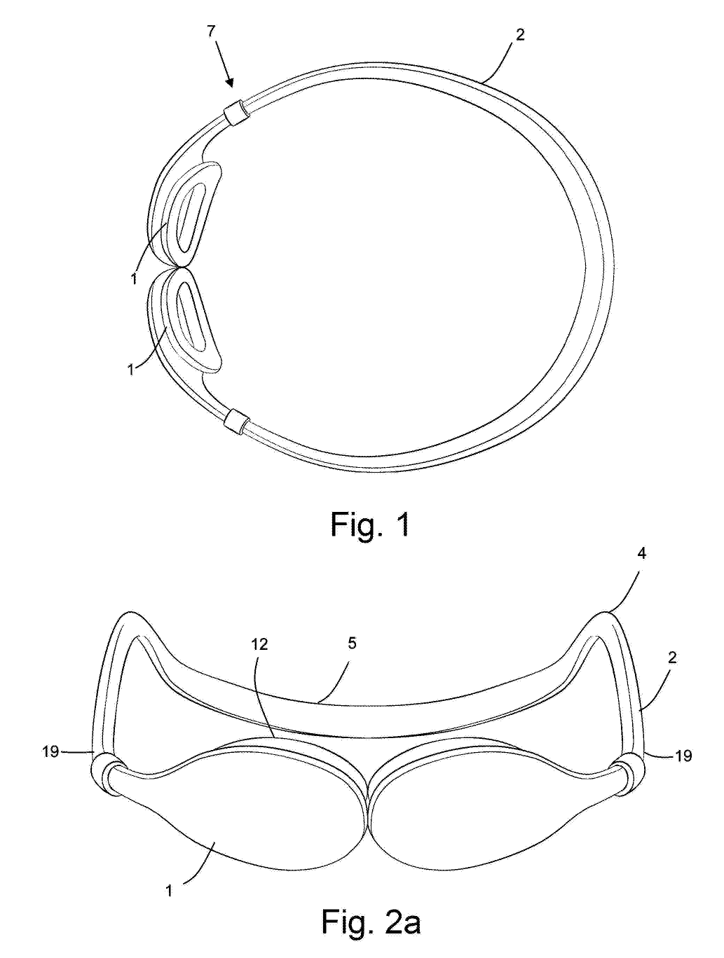

FIG. 1 shows the arcuate arm 2, dimensioned to match the shape of a user's head in the form of an open loop. The open loop is sprung so as to return to the original shape arrangement after being flexed in order to fit around the user's head.

The arm 2 is formed from a lightweight synthetic plastic.

Each distal end 7 of the arm 2 attaches to a screen 1. Therefore both screens 1 are connected to the same arm 2.

The device includes an arm 2 that bows out around the user's temples and touches the user behind the ears in use to ensure it does not cover any of the skin between the hairline and eyelids as this would produce tanning lines.

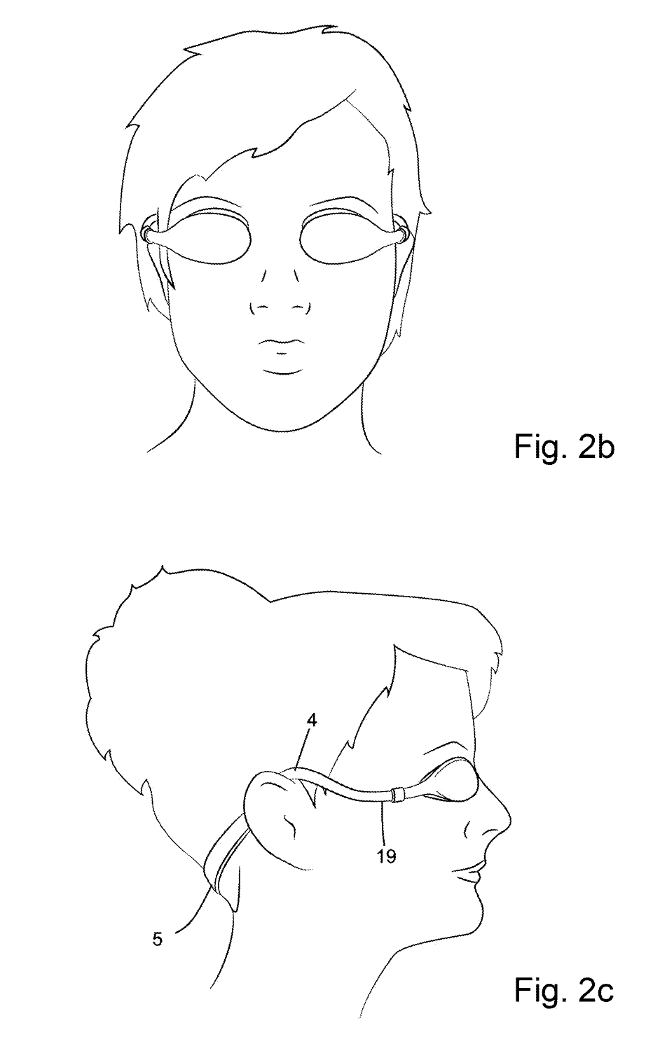

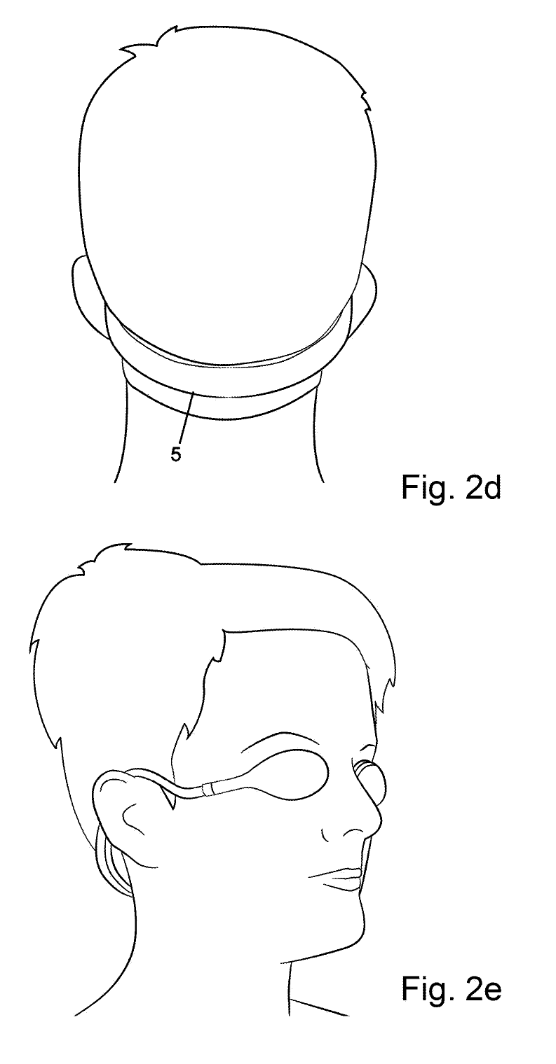

FIG. 2a reveals a front view of the eye covers. Each screen 1 has a smooth outward facing finish that is arcuate so as to match shape of the eye. The screens 1 are teardrop shaped, tapering towards the user's temples. FIGS. 2b to 2e provide various views of the eye cover device of FIG. 2a, being worn by a user.

The arm 2 has an arch 4 where the arm passes over the ear so that the arm 2 is in contact with part of the user, even if the screens 1 are not covering the eyes. The arm 2 includes a dropped portion that drops away behind the arch 4 to follow to the nape of the neck. The dropped portion 5 is the widest part of the arm, fitting flush against the user's head.

When viewed from the front as in FIG. 2 (without the presence of the user) the arm may be seen to comprise side portions 19, each connecting a respective screen 1 to a respective arch 4. In use, each arch 4 is positioned over an ear of the user. Assuming up and down to be defined with reference to the user, the dropped portion 5 extends down from each arch 4 to a lowest point or lowest region at a midpoint of the arm 2.

As may be seen in FIG. 1, the dropped portion 5 of the arm 2 has an arcuate shape when viewed from above. The arcuate shape may fit to the nape of a user's neck (the alignment with the nape of the neck being demonstrated in FIG. 2). The dropped portion 5 may be curved in such a way as to match the back and sides of the user's head. The arcuate shape of the dropped portion 5 may provide a comfortable fit when the eye cover device is in use.

Furthermore, the arcuate shape of the dropped portion 5, when viewed from above, may also provide a comfortable fit when the user, in order to carry the eye cover device, is wearing the eye cover device around the base of his or her neck. When not in use, the user may lower the eye cover device. The user may wear the eye cover device with the arm 2 passed around the base of the user's neck such that the screens 1 rest forward of the user's neck, for example resting on the user's collarbones.

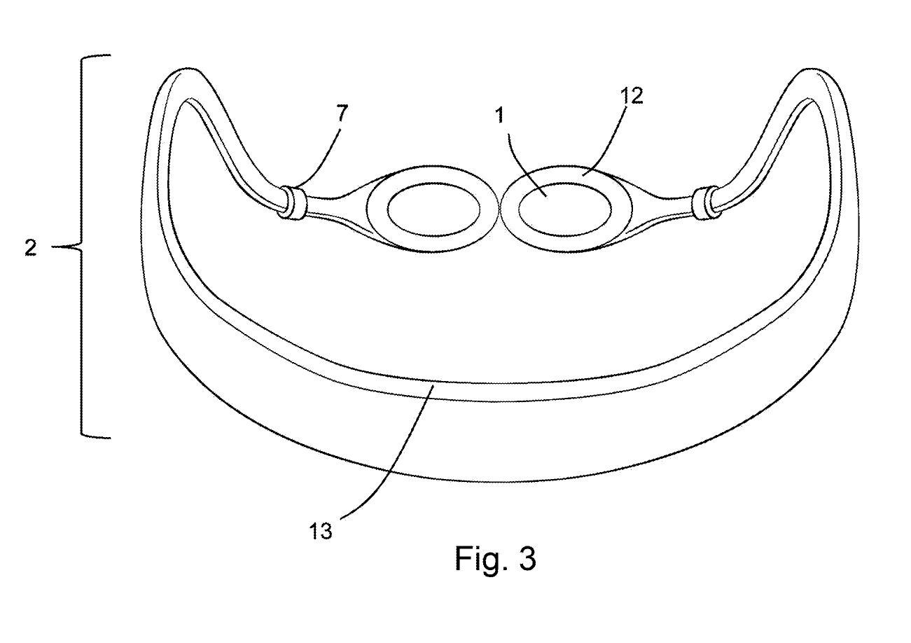

FIG. 3 shows a rear view of the eye covers. The arm 2 has a dropped portion 5 so as to follow the nape of the neck when worn. In some embodiments, the arches 4 may extend higher than where the screens are orientated in use so as to pass up and over the ears. Each distal end 7 of the arm 2 is shown wherein each screen 1 connects to the arm 2 at a respective distal end 7. The peripheries (e.g. peripheral portions) 12 are oval shaped extending around the outer rim of each screen 1.

Padding 13 is provided on an inner face of the arm on the dropped portion 5 so as to improve comfort when worn.

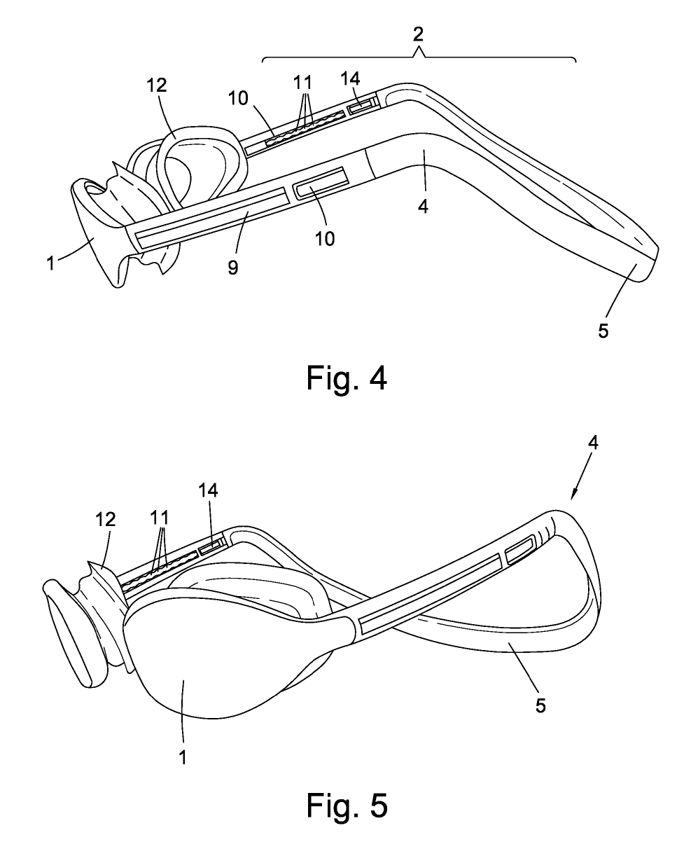

FIG. 4 discloses the side view revealing the arch 4 for each side of the head and the dropped portion 5.

The arm 2 comprises two side portions 19. When viewed from the side (such that the two side portions 19 are overlaid) the side portions 19 are substantially straight (when viewed from above as in FIG. 1, the side portions 19 are curved around the head of the user). In the embodiment of FIG. 4, each side portion 19 comprises a hollow member 9 which connects with the remainder of the arm 2.

A rearward portion of the arm 2 (in this embodiment, dropped portion 5) is inclined downwardly at an angle to the side portions 19. The angle is large enough to position a midpoint of the rearward portion at or near the nape of the user's neck.

The hollow member 9 is shown integrated with the screen 1 being formed as one part. Cutaway portions 10 reveal the arm 2 within the hollow member 9 so as to show the user how far the arm 2 is inserted into the hollow member 9.

The ridges 11 on the arm 2 that enable incremental adjustment of arm length are show through a cutaway 10.

A concertina peripheral portion 12 is shown attached to an inner face of each screen 1. In use, an engagement surface 15 of the peripheral portion 12 is fixed against the user's eye (for example, the engagement surface 15 connects with the skin around a user's eye socket). The engagement surface 15 may be fixed by a suction fit, which may be described as a gentle suction fit. Alternatively or additionally, the engagement surface 15 may be fixed at least in part by surface tension or other surface interaction between the material of the engagement surface 15 and the skin, due to material properties of the engagement surface, for example following pressing of the engagement surface 15 against the skin.

The peripheral portion 12 comprises a concertina fold 16. The peripheral portion 12 comprises a concertina fold 16. The peripheral portion 12 further comprises a channel 17 traversing the peripheral portion 12 in some embodiments, such that air may be admitted into the cavity formed by the user's eye, the peripheral portion 12 and the screen 1.

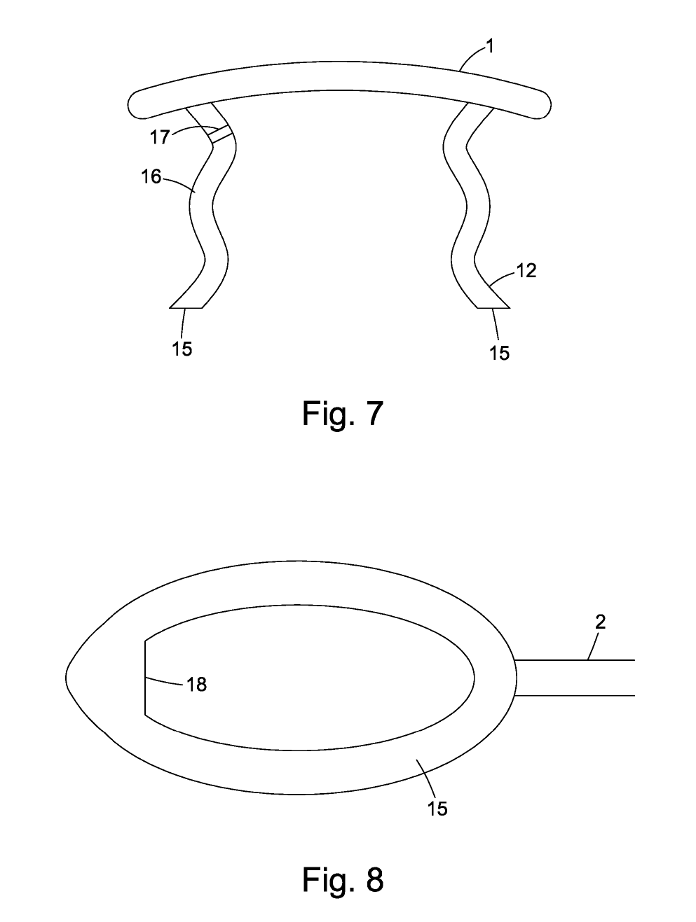

FIG. 7 is a schematic cross-sectional drawing (not to scale) of a peripheral portion 12 attached to an inner face of a screen 1 according to an embodiment. An engagement surface 15 makes contact with the user's eye region (for example, contacts the skin surrounding the eye, eyelid and caruncle), forming a cavity.

Channel 17 is a small hole placed at the base of concertina fold 16 (the part of the concertina fold 16 that is nearest the screen 1), which passes between the outside and the inside of the peripheral portion 12, allowing for a flow of air from the outside to the inside in use, the inside being the part that forms the cavity.

The channel 17 is placed at the part of the peripheral portion 12 that is near to the nose of the user. In other embodiments, the channel 17 may be placed at any suitable location on the peripheral portion 12, for example proximal to the location at which the arm 2 attaches to the screen 1.

When the user puts on the eye cover device, the user may press the peripheral portion 12 towards the eye (for example, by pressing on the screen 1 or by flexing the arm 2). When the user presses the peripheral portion 12 against the eye, the concertina fold 16 may be compressed. When the user stops applying pressure, the concertina fold 16 may relax, which may cause a small decrease in pressure in the cavity formed by the peripheral portion 12, the screen 1 and the eye, thereby facilitating the formation of a suction fit.

The peripheral portion 12 is formed of a soft, resilient material. The use of soft, resilient material may allow the shape of the peripheral portion 12 to adapt to the face shape of different users. The concertina fold 16 may assist in the adaptation of the shape of the peripheral portion 12 to provide a good fit to the face of the user.

In other embodiments, any number of concertina folds 16 may be used. The concertina folds 16 may be substantially parallel to a plane passing through the outer rim of the screen 12.

When the user wishes to remove the eye cover device, the user may apply a pulling force to each peripheral portion 12, for example by pulling on the screen 1 or by flexing the arm 2. When a screen 1 is pulled away from the user's eye, the concertina fold 16 may expand and air may pass more easily through channel 17 than when the concertina fold is relaxed. The seal between the engagement surface 15 and the eye region may thereby be reduced, making it easier to detach the peripheral portion 12.

By providing a channel 17 or breathable portion in the peripheral portion 12, the peripheral portion 12 may be prevented from forming too strong a seal, for example a seal that is strong enough to cause the user difficulty in removing the engagement surface 15 and/or a seal that is strong enough to cause damage to the user's skin (particularly the delicate skin of the periorbital region) when removing the engagement surface 15. The channel or breathable portion may prevent too large a pressure difference to build up between the two sides of the screen.

In some embodiments, the channel may be formed such that in use it is substantially closed unless a pressure difference between one side of the screen and the other becomes sufficiently large causing the channel to open and the pressure difference to reduce. For example, in some embodiments the channel may be formed such that opposing walls of the channel rest in contact with each other, closing the channel, unless the pressure difference becomes sufficiently large to force the walls apart and open the channel.

FIG. 5 reveals an isometric view of the device. The peripheral portion 12 serves to distance the screen 1 from the eye so as to allow blinking. The arm 2 is narrower at the temples and arch 4 but widens at the dropped portion 5.

FIG. 8 shows a view of an engagement surface 15 of a peripheral portion 12, also showing the position of part of the arm 2 with relation to the engagement surface 15. The engagement surface 15 is viewed in the direction looking outwards from the user's eye.

The engagement surface 15 has the shape of a generally oval ring. The engagement surface 15 has an inner edge having a generally oval shape, but comprising a straight portion 18 at the nose end of the oval (the end that is distal to the arm 2). The straight portion 18 may facilitate a good fit to the user's nose.

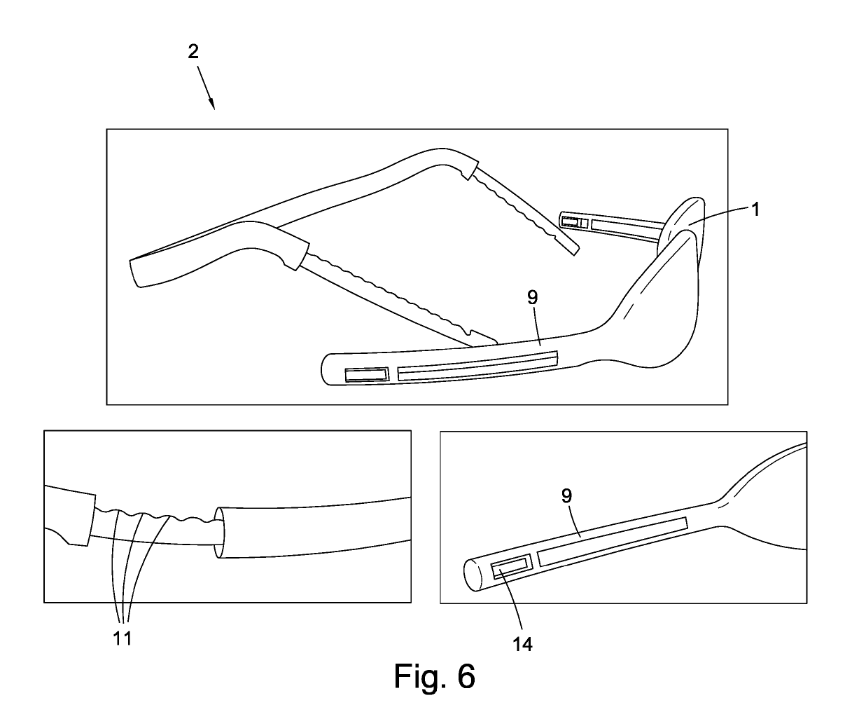

FIGS. 6A, B and C shows different views of the hollow member 9 and arm 2. FIG. 6A shows the arm detached from the integrated hollow member 9 and screens 1. A plurality of ridges 11 are shown on the arm 2.

Cutaways 10 are provide on inner and outer faces of the hollow member 9. The catch 14 is shown on the hollow member 9 which serves as a ratchet to enable the arm 2 to be incrementally moved through the hollow member 9.

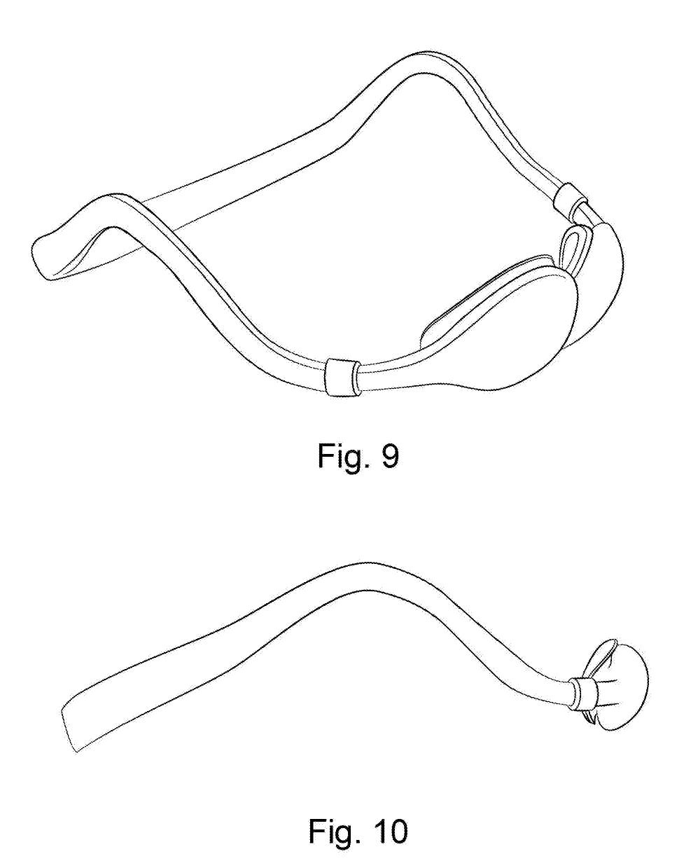

FIG. 9 provides a further view of an embodiment of the eye cover device. FIG. 10 shows a view of part of an arm of an eye cover device according to an embodiment.

The devices illustrated in FIGS. 1 to 10 may be produced in a range of different sizes, for example in adult and child versions.

The device has been described in relation to its use in sunny environments and/or to allow tanning. The device may also be used primarily as a sleep, relaxation and/or concentration aid. For instance, the device may be used during travel on an aircraft or other form of transport, to allow the user to rest, relax or sleep by blocking light to the eyes. The device may be used as a sleep, relaxation or concentration aid in any other environment. The channel or breathable fabric features may be particularly useful in an aircraft or other travel environment where significant pressure changes may be experienced by the user.

The device may be used as a therapeutic aid, for example in eye treatment methods or relaxation methods, and can provide an effective way of stopping light to the eye without putting pressure on the eye or eyelid itself. The device may for example be used in conjunction with the Bates Method for improving eyesight

The invention has been described by way of examples only and it will be appreciated that variation may be made to the above-mentioned embodiments without departing from the scope of invention.

1-42. (canceled)

43. An eye cover device comprising: a pair of screens, each screen is dimensioned to cover an eye, both screens are connected to an arm that in use passes around a rear portion of a user's head connecting the screens together, wherein the screens are displaceable by an arm mechanism.

44. A device according to claim 43 wherein the arm is a curved elongate length of material arranged in use to be in contact with the user's head.

45. A device according to claim 43 wherein the arm is an open loop.

46. A device according to claim 43 wherein the arm includes arches for passing over and resting upon the user's ears.

47. A device according to claim 43 wherein the arm comprises two side portions and a rearward portion inclined downwardly at an angle from the side portions.

48. A device according to claim 47, wherein, in use, the rearward portion extends downwards from the user's ear to the nape of the user's neck.

49. A device according to claim 43 wherein the rearward portion comprises a dropped portion so as to position the arm at the nape of the user's neck so as avoid discomfort when the user places their head against a headrest.

50. A device according to claim 43 wherein at least one of a) or b):
a) the arm is sprung in order to exert gentle pressure against the user's head to hold the device securely in position whilst worn;
b) the screens are attached to distal ends of the arm.

51. A device according to claim 43 wherein at least one of a), b) or c):
a) the arm mechanism includes a means of adjusting arm length;
b) a hollow member attached to each screen receives the arm and enables adjustment;
c) the distal end of the arm is hollow and capable of receiving a concentric member that is attached to the screens to enable adjustment of arm length.

52. A device according to claim 43 wherein the arm mechanism includes a hinge for displacement of the screens, wherein optionally:
the hinge is positioned at the distal end of the arm.

53. A device according to claim 43 wherein positioning of the screens by the arm mechanism is user settable.

54. A device according to claim 53 wherein at least one of a), b), c) or d):
a) each screen is user settable by means of a ratchet;
b) the arm includes anti slip grips to assist in keeping the device in place whilst being worn;
c) the arm includes adjustable length parts;
d) the device includes transparent pieces so as to be discreet in use.

55. A device according to claim 43 wherein each screen includes or is associated with a peripheral portion.

56. A device according to claim 55 wherein at least one of a), b), c), d), e), f) or g):
a) the peripheral portion is a resiliently deformable pad or cowl;
b) the peripheral portion comprises at least one fold;
c) the peripheral portion comprises an engagement surface for making contact with an eye region of the user;
d) an inner edge of the engagement surface comprises a substantially straight portion at an end of the engagement surface that, in use, is proximal to the nose of the user;
e) the peripheral portion comprises a channel and/or a breathable piece;
f) the peripheral portion includes eye treatment means;
g) the peripheral portion is capable of receiving an eye treatment lotion/ointment/liquid/gel/solution.

57. A device according to claim 43, comprising, for at least one of the screens, means for reducing a difference in pressure between one side of the screen and another side of the screen when in use.

58. A device according to claim 43 including a bimetallic arm wherein temperature changes cause mechanical displacement.

59. A device according to claim 43 wherein at least one of a), b) or c):
a) each screen is translucent;
b) each screen is a lens;
c) the device is capable of communicating with a remote unit.

60. A device according to claim 43 wherein a sensor is provided to detect variables including: ambient conditions, from the group comprising, light intensity, temperature, humidity, proximity of persons, and optionally wherein at least one of a), b) or c):
a) the sensor is capable of detecting light;
b) the sensor is capable of detecting temperature;
c) the sensor is configured to communicate with a transmitter which is capable of transmitting data relating to the detected variable to the remote unit.

61. A device according to claim 43, wherein at least one of a), b), c) or d):
a) the device includes an alert;
b) the device includes an alert and the alert includes a vibrating means;
c) the device includes an alert and the alert is audible;
d) The device includes an alert and the alert is visual.

62. A method of providing eye coverage using the device according to any of claim 43.
